(19) United States
(12) Patent Application Publication
(10) Pub. No.: US 2023/0077642 A1
(43) Pub. Date: Mar. 16, 2023

(54) Systems and methods for performing Correlated Multiphasic Analysis

(71) Applicant: Arun Christopher Konanur, London (CA)

(72) Inventor: Arun Christopher Konanur, London (CA)

(21) Appl. No.: 17/470,321

(22) Filed: Sep. 9, 2021

(51) Int. Cl. G16B 20/00; G16B 50/30; G16H 50/70

(52) U.S. Cl. CPC G16B 20/00; G16B 50/30; G16H 50/70

(57) ABSTRACT

A bioinformatic system that identifies the common ancestral origins of otherwise uncorrelated autosomal DNA (atDNA) matches is disclosed. The invention consists of three main components: The first is Correlated Multiphasic Analysis (CMA) a process of logically associating subsets of In Common With (ICW) atDNA matches in order to arrive at a solution set for queries investigating ancestral family lines. The second is a set of automated scripts, formulae, and data structures to facilitate desktop correlation and tabulation utilizing CMA in conjunction with a desktop spreadsheet program such as Microsoft Excel. The third is a system of data tables and methods to facilitate CMA within a database management system (DBMS) at the enterprise level.

### FIELD OF THE INVENTION

The present invention relates to a system that performs Correlated Multiphasic Analysis (CMA), a method of organizing autosomal DNA matches, both on a personal (desktop spreadsheet tabulation) and on an enterprise (database management system) platform.

### BACKGROUND OF THE INVENTION

Direct-to-consumer autosomal DNA (atDNA) testing for the purpose of ancestry analysis was introduced in 2007, and since then millions of consumers have purchased test kits from one or more commercial entities which offer this service (23andMe, AncestryDNA, Family Tree DNA, MyHeritage, etc.). In each case, an individual's atDNA is sampled along roughly 700,000 single-nucleotide polymorphisms (SNPs), which are in turn compared against the test results of other customers of that same service (as many as 20 million other tests depending on the service), in order to generate a list of member matches—generally presented as a list of member names and/or test kit numbers, sorted by linkage—the number of DNA units shared between the test subject and a given member. The unit for the tabulation of segments of corresponding atDNA is the centiMorgan (cM).

Conventional methods for analysis of atDNA matches involve surveying matching members' family trees for common individuals or surnames in order to determine a Most Recent Common Ancestor (MRCA) through which the test subject and their member match are descended. At best, this may be feasible for 1 to 1.5% of all member matches. Supplementary techniques, such as clustering matches which share DNA segments with known MRCA matches, may elevate the number of members associated with identified ancestral lines to the range of 3 to 5%. Granular methods of DNA analysis, which delve into the structures and correspondences within chromosomes, can yield insights into close relations within endogamous communities, but are limited as to their ancestral reach.

The remaining 95% of atDNA matches tend to remain unidentified because of missing or inaccurate family trees, non-paternity events (otherwise known as NPEs: instances where the genealogical record departs from the genomic line), or because the amount of atDNA in common (known as shared linkage) falls below a workable threshold (typically 40 cM). Correlated Multiphasic Analysis (CMA) addresses these impediments by evaluating the associative properties of atDNA test results across the gamut of a subject's matches and by indexing an individual match across multiple scenarios, grouping correspondences into functional equivalence classes derived (and/or inferred) from verified MRCA relationships.

### SUMMARY OF THE INVENTION

This invention is directed to address the limitations of traditional analytical practices, as outlined in the preceding background section. To this end, CMA delivers powerful insights drawn from the totality of a subject's atDNA results, rather than the top 1 to 5% of matches, and correlates member matches beyond the reliable 5-6 generation/200-year window otherwise available through segmental analysis of atDNA. CMA is dynamic and multiphasic, reframing its solutions as additional member matches and/or correlating criteria are added. CMA quickly identifies NPEs—test subjects and associated data which do not correlate—without impacting the quality of its core findings, and supports intuitively structured queries, accessible to anyone with an appreciation of the concept of ancestral family lines and common ancestors.

When deployed at the enterprise level, CMA leverages large sets of atDNA matches, with or without associated family trees. CMA does not require any additional processing of raw atDNA data, nor does the CMA process assume any advanced scientific knowledge on the part of the end user. CMA rewards the targeted testing of extended family members and lends itself to an interactive click-driven interface.

CMA can specifically address the genealogical “brick wall” challenges faced by individuals with unknown parentage, or immigrant ancestors whose records from their home countries may be incomplete or inaccessible. CMA's ability to correlate ancestral lines beyond a 200-year horizon makes the process particularly useful to, among others, African-Americans and other marginalized populations, whose ancestors might not appear by name on US censuses prior to 1870.

In addition to correlating the atDNA matches of test subjects of known ancestry, CMA can impute a genealogical relationship by comparing the patterns, correlations and correspondences of an unknown test subject's atDNA matches with those of known genealogical relations.

The CMA process may also be applied to DNA chains other than atDNA, including Y-DNA, and mitochondrial DNA (mtDNA). Beyond an exclusively genealogical purview, CMA may be applied in the field of medicine, as a Correlated Multiphasic Analysis of atDNA matches from individuals bearing specific gene-linked traits or conditions would allow clinicians to generate broad subclasses of at-risk individuals with potentially greater or lesser susceptibility to specific viral infections or hereditary conditions, and to fine-tune these projections as additional individuals or populations are tested. Other biomolecules such as protein chains, RNA and mRNA may also be correlated using CMA. Additionally, CMA may be applied to the pedigrees of species other than humans—including, but not limited to: bacteria, viruses, purebred dogs, and thoroughbred horses.

### BRIEF DESCRIPTION OF THE DRAWINGS

In order to facilitate a fuller understanding of the present invention, reference is now made to the accompanying drawings. These drawings should not be construed as limiting the present disclosure, but are intended to be exemplary only.

FIG. 1 is a process flowchart illustrating Correlated Multiphasic Analysis (CMA). Each sub-process has been numbered for reference; references are maintained throughout the detailed description of the invention.

FIG. 2 illustrates the concept of Most Recent Common Ancestor (MRCA), a genealogical concept of universally regarded value.

FIG. 3 illustrates how the MRCAs of a collection of two or more individuals also define a larger associative framework, the genetic complex ( [symbol] )—a construction specific to CMA.

FIG. 4 illustrates that a complex defined by D—a distant relation common to A and B—is a proper subset of the complex formed by [symbol] _{MCRA(A,B)}.

FIG. 5 illustrates that a complex defined by E—a less distant relation from a line other than D— is disjunct with respect to [symbol] _{MCRA(A,D)} and less specific.

FIG. 6 is an overview of the tripartite structure of the CMA Master Workbook, a desktop implementation of the CMA process.

FIG. 7 is a diagram of the Correlation Worksheet section of the CMA Master Workbook, illustrating areas of user input, computational formulae, and scripted interface buttons.

FIG. 8 presents a sample pedigree and its corresponding entries in the Summary Module's Table of Complexes.

FIG. 9 presents the interface button VBA scripts from the Correlation Worksheet alongside a shared subroutine method for populating the analytic core set of the Summary Module.

FIG. 10 is a diagram of the rightmost area of the Correlation Worksheet, illustrating how the formulae that flag potential additions to the analytic core set of evolve as additional test subjects participate in the CMA process.

FIG. 11 is a diagram of the Tabulation Matrix of the CMA Master Workbook, illustrating three instances of the computational formulae used to cross-reference 20,000 members of the analytic core set against 26 test subjects.

FIG. 12 is a diagram of the Summary Module of the CMA Master Workbook, which includes the Table of Complexes (TOC), and a CMA Summary that collates and interprets the findings of the Tabulation Matrix, navigable via scripted sortation buttons.

FIG. 13 presents the VBA sortation code for the Summary Module of the CMA Master Workbook.

FIG. 14 is a diagram overview of the DBMS tables and relations required to perform CMA at the enterprise level.

### DETAILED DESCRIPTION OF THE INVENTION

I. CMA Process

Correlated Multiphasic Analysis formulates its solutions by applying unary operations—primarily union (∪), intersection (∩), and complementation (˜)—to an analytic core set (or ACS, designated by ϑ) of atDNA matches subtended by genetic complexes ( [symbol] ) derived from shared ancestral lines.

The analytic core set (variously, ACS or ϑ) is central to the CMA process and is essentially the set of all correlated matches of cardinality 2 or greater. The ACS is employed as an axis of comparison across multiple atDNA test subjects, and the analytic core set's membership will necessarily increase as additional atDNA member matches are correlated. The ACS is partitioned into equivalence classes labelled by the Most Recent Common Ancestors (MRCAs) associated with the genetic complexes formed by the atDNA matches correlated by the CMA process—the end result being that CMA provides the researcher with collections of atDNA matches that exhibit common properties of inheritance across multiple verifiable criteria, effectively saying, “Search here, and you will find the answer you seek.”

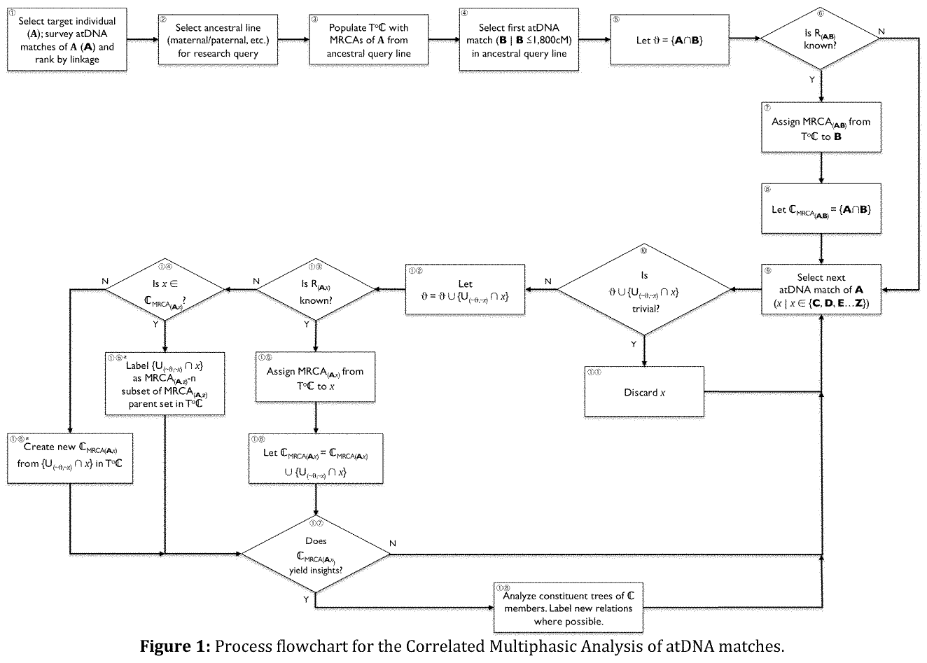

FIG. 1 is a process flowchart illustrating CMA. Each sub-process has been numbered for reference:
\textcircled{1} The Target Individual is the focal point of the CMA process and the locus about which the correlation of atDNA initially occurs. The Target will have obtained the results of an atDNA test and, insofar as is possible, has assembled a pedigree chart of their ancestral lines. The set of atDNA matches for the target individual A is indicated as A.

Most providers of atDNA tests report their results as a list of member matches ranked by linkage, or the amount of DNA shared by the test subject and each member match. To facilitate the selection of member matches for correlation, the Target Individual's matches should be ranked in this manner. Where possible, it is useful to identify known genealogical relations among the target individual's atDNA matches, both by the type of relationship, as well as maternal/paternal valence and the relevant family line. “Paternal second cousin once removed (2C1R) via Jones line” is an ideal example.
\textcircled{2} To simplify and bring focus to an investigation, it is generally recommended that the CMA process should be directed along a single maternal or paternal ancestral line, but in principle both lines may be used, particularly when exploring endogamous ancestry. The best CMA queries are those which target an ancestral “brick wall” where other analytic methods have failed to produce viable leads. In these cases, CMA's ability to delineate interrelated sets of atDNA connections with similar properties generates fresh leads that can be further investigated via traditional genealogical methods.
\textcircled{3} Where a genealogical relationship is known to exist between two individuals, we can identify their Most Recent Common Ancestor (MRCA) as the point at which their shared ancestral lines diverge. Full genealogical relations will share an MRCA couple, whilst half-relations will have an individual in common. However, if we know the parents of the half-relation's MRCA, that individual common ancestor may likewise be identified in terms of their parents—an MRCA couple themselves. Surnames are typically used to identify an MRCA ancestral couple, so if the common ancestors of A and B are John Smith and Mary Jones, we may write MRCA_{(A,B)}=[Smith-Jones].

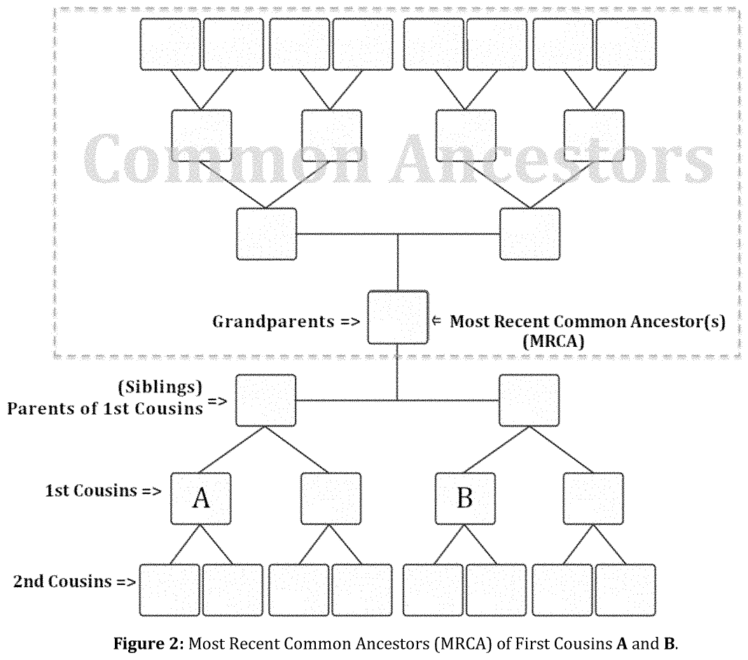

FIG. 2 illustrates how two first cousins (A and B) share an MRCA set of grandparents. It should be noted that, in addition to sharing a set of a grandparents, A and B also share each and every ancestor in their common ancestors' pedigree. Genetically speaking, even if an MRCA is unknown, common ancestral lines exist between any two individuals who share DNA in excess of a trivial threshold—say, 6-10 cM. The MRCA relation is reflexive, a property which will be explored in analyzing the genetic complexes ( [symbol] ), which subtend the analytic core set (&).

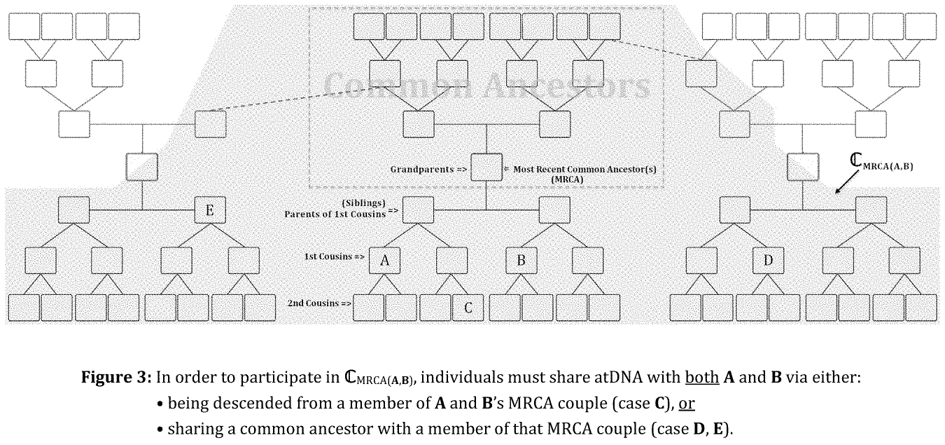

FIG. 3 illustrates that any individual whose atDNA test matches both A and B must be connected to the MRCAs of A and B—either as a direct descendant of at least one member of that MRCA couple (hypothetical C) or through an ancestor found among the MRCAs' pedigree (hypothetical D or E). The set of all individuals that share an atDNA match with both A and B are said to form a genetic complex ( [symbol] ) about A and B, notated as [symbol] _{(A,B)} or more generally by using the surnames of MRCA_{(A,B)}, such as [symbol] _{[Smith-Jones]}. Connections to [symbol] _{MRCA(A,B)} exist in the manner illustrated for hypotheticals D and E from every individual within the “Common Ancestors” group, so the genetic complex is more diffuse than can be easily illustrated in one panel, but given the trillions of potential connections among even a few million atDNA test subjects, the ability to refer to the set of all members which match both subjects A and B is of great functional utility.

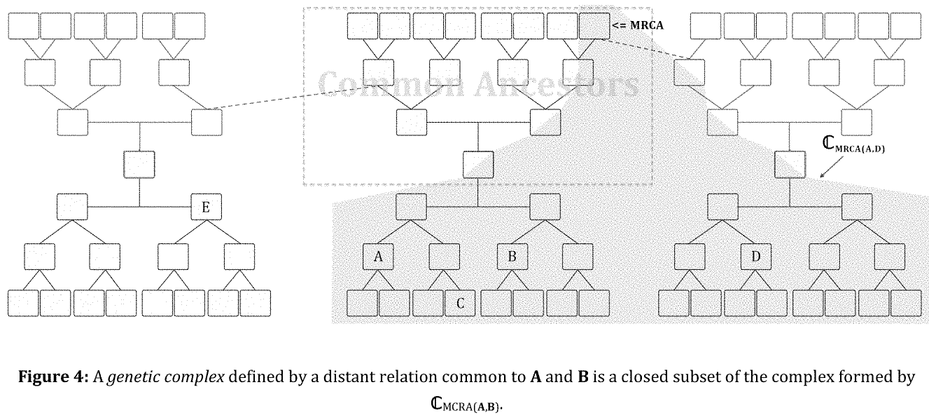

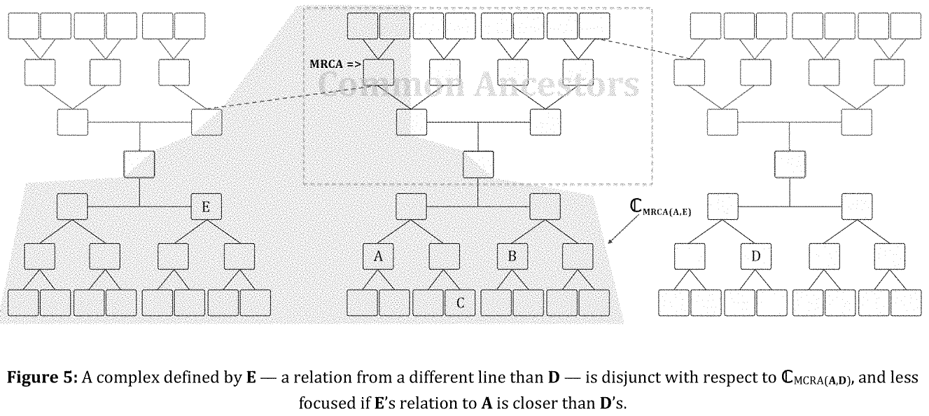

It should be emphasized that hypotheticals C, D, and E are precisely that: generalized placeholder individuals without a defined genealogical relationship to A and B. If hypothetical C were in fact A's neice/nephew or B's 1^{st} cousin once removed, the impact on [symbol] _{MRCA(A,B)} would be minimal, as C already shares the same MRCA as A and B. However, if hypotheticals D or E were actually related to A and B in the manner illustrated, their MRCAs would form distinct complexes about the ancestors each has in common with A and B. This recontextualization in the presence of newly identified genealogical relationships goes the heart of the multiphasic properties of CMA and testifies to the adaptability of the process. FIGS. 4 and 5 illustrate these alternate complexes: ≤_{MRCA(A,D)} and [symbol] _{MRCA(A,E)}.

FIG. 4 shows that if D is more distantly related to A than B is to A, and if MRCA_{(A,D)}=MRCA_{(B,D)}, then [symbol] _{MRCA(A,D)} will be a proper subset of [symbol] _{MRCA(A,B)}. Because the genetic complexes of distant MRCAs yield more focused collections of ancestors, it stands to reason that when assigning a complex to a member match shared by several test subjects, we should regard any matches with test subjects with more distant MRCAs relative to A as defining which complex the member match is assigned to, even—and especially—if other subjects with closer MRCAs also participate in that complex. It is for this reason we number the MCRAs in our table of complexes in terms of ascending generations removed from our target individual, A.

FIG. 5 illustrates that a genetic complex formed by subjects A and E will be disjunct from from [symbol] _{MRCA(A,D)} if D and E are not from the same ancestral lines, even though both share atDNA with A and B. This has profound implications and explains CMA's ability to stratify and differentiate various ancestral lines. Because MRCA_{(A,E)} is a closer relation to A than MRCA(A,D), the complex about A and E is less focused (i.e. more diffuse and potentially contains a larger number of individuals) than [symbol] _{MRCA(A,D)}.

A table of complexes (T° [symbol] ) organizes and tallies the atDNA matches of the analytic core set (ϑ) according to their membership in a particular complex. The simplest and most comprehensive way to structure this table is to list all known MRCA couples from the Target Individual's pedigree.

For the test subject A, the immediate MRCAs associated with A's T° [symbol] are:

| | Generations |
|---|---|
| MRCA couple | removed from A |
| Child(ren) of A and their spouses | −1 |
| A - spouse of A | 0 |
| A's parents | 1 |
| A's maternal grandparents | 2 |
| A's paternal grandparents | 2 |
| A's maternal great-grandparents (two distinct sets) | 3 |
| A's paternal great-grandparents (two distinct sets) | 3 |
| A's maternal great-great-grandparents (four distinct | 4 |
| sets) | |
| A's paternal great-great-grandparents (four distinct | 4 |
| sets) | |
| A's maternal GGG-grandparents (eight distinct sets) | 5 |
| A's paternal GGG-grandparents (eight distinct sets) | 5 |

In practice, most CMA inquiries will investigate either a maternal or paternal line, so the number of MRCA complexes for generations 2 and greater will be halved. Further, by restricting an investigation to matches of 1,800 cM or less, generation 0 and those adjacent to A are removed from consideration.
\textcircled{4} Because they tend to produce trivial associations, the CMA process discourages correlating parent or sibling atDNA matches. However, these matches may be employed when investigating instances of unknown or uncertain parentage. Otherwise, an investigation typically begins with the selection of the closest match of 1,800 cM or less associated with the Target Individual's maternal or paternal line under investigation. This member may be a half-sibling, aunt/uncle, grandparent, a first cousin of the Target Individual—or a more distant relation—depending on the roster of A's member matches. This individual is designated as B.
\textcircled{5} The analytic core set ( [symbol] ) is the set of all atDNA member matches participating in a given CMA whose cardinality amongst our test subjects is greater than 1. For the starting sets A and B, [symbol] equals {A∩B}. [symbol] is augmented by process 00 each time an additional set of atDNA matches is analyzed.
\textcircled{6} The genealogical relationship between individuals A and B is indicated as R_{(A,B)}. A genealogical relationship is required in order to determine the Most Recent Common Ancestor (MRCA) of A and B but will not otherwise affect the CMA findings.
\textcircled{7}, \textcircled{8} Subject B—and by extension, the atDNA matches of subject B in common to A—is assigned to the genetic complex ( [symbol] ) corresponding to the MRCA couple shared by A and B: [symbol] _{MRCA(A,B)}.

The genetic complex of A relative to B is written as [symbol] _{(A,B)} and is commutative, so [symbol] _{(B,A)} is functionally the same as [symbol] _{(A,B)}. [symbol] _{(A,B)} includes all descendants of A and B's common ancestors—in principle, even those which might not match both A and B—and also all of A and B's “complex cousins”: tested members which match both A and B, even if their exact genealogical relationship is unknown.

Because all of A and B's In Common With (ICW) matches must connect in some way to the MRCA of A and B, we can state that [symbol] _{(A,B)} is identical to [symbol] _{MRCA(A,B)}. The reflexive nature of the genetic complex suggests that if we analyze the atDNA matches of another individual, C, that shares the same MRCA with A and B, we can state with confidence that [symbol] _{(A,C)}, [symbol] _{(A,C)}, and [symbol] _{(A,B,C)} will also be identical to [symbol] _{MRCA(A,B)}. It follows that if [symbol] _{MRCA(A,B)} were to encompass several test subjects with a common MRCA—say A, B, C, D, and E—then [symbol] _{MRCA(A,B)} would equal [symbol] _{P(A,B,C,D,E)} where P(A,B,C,D,E) represents all non-trivial (2 element and greater) combinations and permutations of elements A through E.

Since these genetic complexes are organized about MRCAs, processes \textcircled{7} and \textcircled{1}\textcircled{3}—“record MRCA_{(A,B)}” and “record MRCA_{(A,x)}”—require only that our table of complexes (T° [symbol] ) should comprise a list of MRCA couples from which we can associate the letter-name of an individual who shares that MRCA couple with A and, for comparative and analytic purposes, a value representing the number of generations that MRCA is removed from the test subject A. These letter-name designations will form the permutation elements alluded to in the preceding paragraph, which are fundamental in constructing equivalence classes of matches to serve as a foundation for a CMA-based solution set.
\textcircled{9} The power and elegance of CMA derives from its ability to quickly and easily correlate sets of atDNA matches from multiple test subjects. Processes \textcircled{9} through \textcircled{1}\textcircled{8} form an iterative cycle, comparing our universal set of all surveyed matches (U) against the atDNA matches of individual x, tallying x according to the MRCA they share with A and augmenting ϑ with any matches external to ϑ that x shares with the universal set.

The selection of atDNA matches for correlation subsequent to match B will necessarily vary with each investigation, but several desiderata are likely to figure prominently:
known genealogical relations of A
atDNA matches sharing significant linkage with A
atDNA matches with extensive family trees verified by quality research and/or DNA
atDNA matches with significant connections to an ancestral line of investigation
atDNA matches whose shared linkage ranks at the top of their genetic complex
\textcircled{10}, \textcircled{1}\textcircled{1} Not every member of A will be a suitable candidate for CMA. In a query along paternal lines, the set {ϑ∩x} of a maternal match (x) will yield a trivial result, matching only A and descendants of A's mother. Similarly, if x is a direct descendant of a previously evaluated member of A, then x will neither augment ϑ nor meaningfully subtend any of ϑ's complexes. In the interest of efficiency these redundancies may be removed from analysis in favor of more relevant data. Conversely, if one is seeking to prove that match x is a direct descendant of another test subject, this is precisely the relationship between test results required to demonstrate this fact.
\textcircled{1}\textcircled{2} The atDNA matches of x—a non-trivial match of A—are compared against the set of all atDNA matches from previously correlated individuals. Any duplicated elements of x not already members of ϑ will be added to ϑ.
\textcircled{1}\textcircled{3} R_{(A,x)}, the genealogical relationship between A and x, is evaluated for the purpose of assigning [symbol] _{(A,x)} to an existing [symbol] _{MRCA(A,x)} hierarchy.
\textcircled{1}\textcircled{4}, \textcircled{1}\textcircled{5}, \textcircled{1}\textcircled{5}^{a} If R_{(A,x)} is known, then x is tallied in the table of complexes under MRCA_{(A,x)}, the MRCA couple x shares with A.

When R_{(A,x)} is unknown, and x is already a member of an existing complex ( [symbol] _{MRCA(A,z)}), then that complex may be regarded as the parent set of {A∩x} and {A∩x} may be designated as [symbol] _{MRCA(A,z)}-n, where n is a natural serial identifier. The case study of the Appendix section illustrates this procedure in its latter half.
\textcircled{1}\textcircled{6}, \textcircled{1}\textcircled{6}^{a} In the event that x represents an outlier match—neither a known relation to A, nor an element of a genetic complex about a known relation—then [symbol] _{MRCA(A,x)} may be provisionally designated as its own equivalence class, pending further analysis. Otherwise the [symbol] _{(A,x)} of known relation will by definition be a subset of [symbol] _{MRCA(A,x)}.
\textcircled{1}\textcircled{7}, \textcircled{1}\textcircled{8} From time to time it may be advantageous to suspend the CMA process in order to survey the wealth of information accumulating in the genetic complexes about ϑ. Such analysis may clarify the relationship of independent complexes created by process \textcircled{1}\textcircled{6}^{a}, or provide guidance in the selection of the next atDNA match in process \textcircled{9}. A close reading of the constituent trees of individuals assigned to a given [symbol] _{MRCA(A,x)} may suggest linkage to a particular ancestral line within the pedigree of MRCA_{(A,x)}—generating a “probable complex” within or even among MRCA complexes. Analysis may also suggest avenues of investigation best served by constructing a new CMA framework focused about a subset of the current ACS (see Appendix). Finally, other methods of genealogical research may be employed to determine the MRCA of atDNA matches that share significant linkage with the Target Individual or other known relations.

II. Personal CMA on the Desktop Computing Platform

CMA formulates its solutions by tabulating the intersection of sets of atDNA matches from individuals of known and unknown genealogical relationship. While this could conceivably be accomplished using pen and paper, the task of comparing upwards of 5,000 to 40,000 atDNA matches per subject across a dozen or more test subjects lends itself to computational analysis. Spreadsheet programs represent one class of widely available tools capable of performing such tasks, with Microsoft Excel the leader in this class of applications.

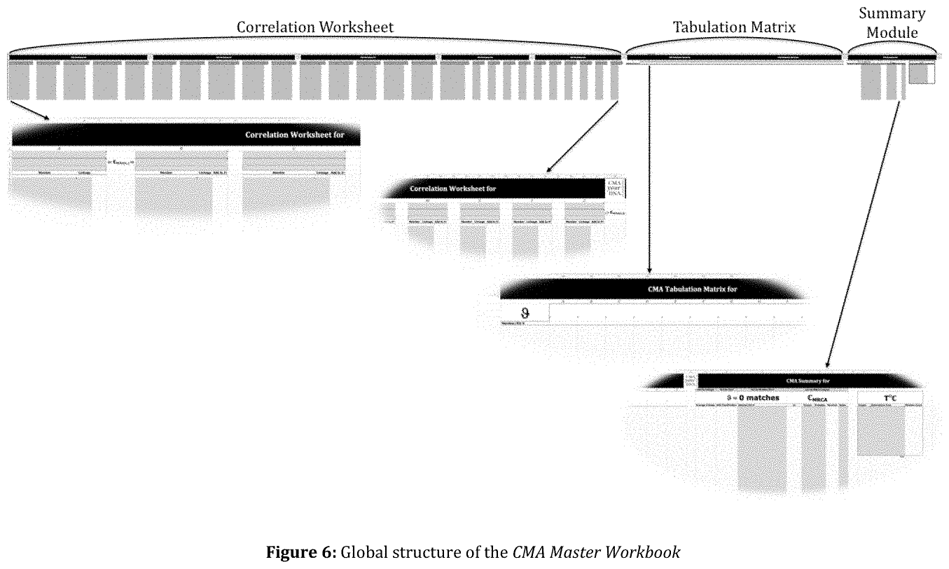

The CMA Master Workbook models the processes of FIG. 1 in a scripted application package in Microsoft Excel. FIG. 6 illustrates the tripartite structure of the CMA Master Workbook: a Worksheet Module, a Tabulation Matrix, and a CMA Summary. The black bar at the top of the sheet identifies the current module and the name of the Target Individual. [CMA your DNA] buttons provide navigational assistance, moving the user rightwards to the next section of the current module, on to the next module, and finally back to the initial home area of the worksheet. Cells with a white ( [symbol] ) background are locked and may contain formulae or calculations, whilst cells with a darker gray background ( [symbol] ) are formatted to receive user input. Light gray ( [symbol] ) cells in the diagrams are actually light blue and contain scripted buttons. In FIGS. 5 through 9, [calculated results] are indicated with [square brackets] in the Geneva font, whilst user supplied data is indicated in italics. Cell references are in parentheses (column→rows↓).

The CMA Master Workbook illustrated herein is configured to correlate as many as 26 test subjects of up to 50,000 atDNA matches each, tabulated across an analytic core set of up to 20,000 data elements. However, these dimensions represent arbitrary parameters based on the probable cardinality of atDNA test results whilst making optimal use of the computational power of the desktop environment, and should not be construed as limiting the capabilities of the CMA process.

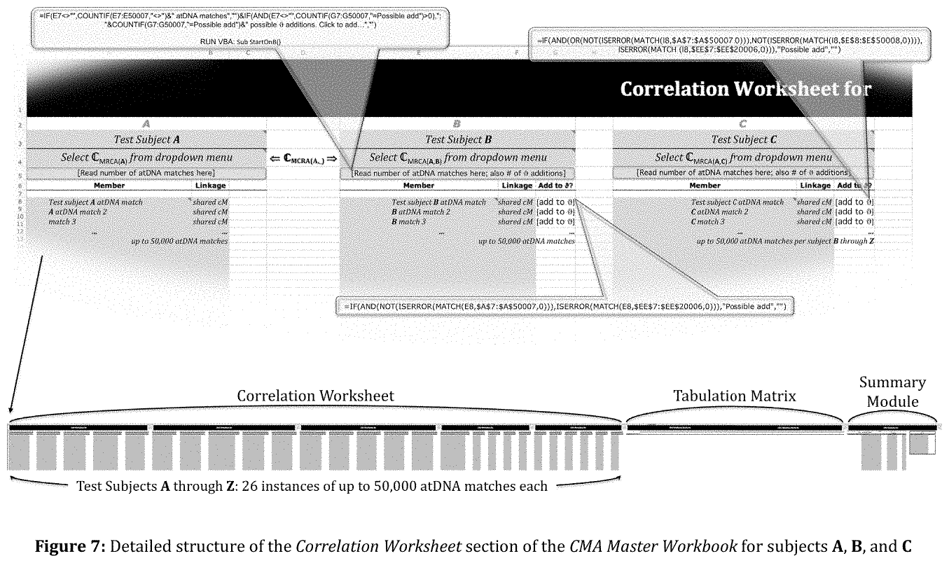

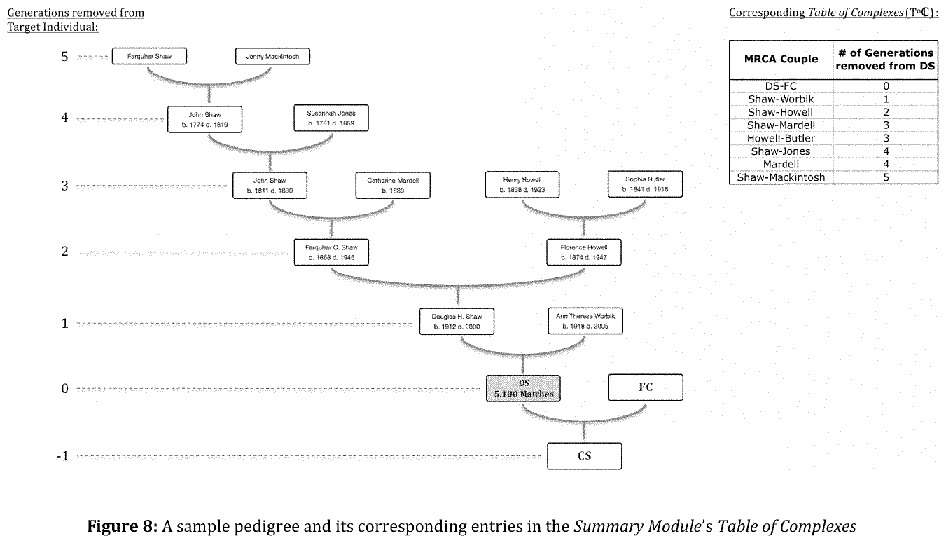

The numbering of processes in the process flowchart of FIG. 1 is maintained in the following description of the structure and operation of the CMA Master Workbook:
\textcircled{1} Opening the CMA Master Workbook displays a message regarding support resources and, if this is the first time the sheet is opened, asks for the name of the test subject A. FIG. 7 shows where A's name is entered (cell A3). Formulae linked to this cell also display A's name in cell (A7) and in the module headings in the black bars at the top of the sheet. The presence of a named Test subject A (through Z) causes the topmost white cell of each subject's Linkage column (B7, F7, J7, etc.) to populate with the word [Self]. The user may then copy and paste up to 50,000 of a test subject's atDNA matches from another excel spreadsheet (or from a delimited text file prepared from the website of whichever testing company A has tested with). The [Read number of atDNA matches here] cell (A5) contains no scripted functions, but will display the number of atDNA matches associated with subject A.
\textcircled{2}, \textcircled{3} After selecting a query from A's maternal or paternal ancestral lines, the user should next populate the Table of Complexes (or T° [symbol] , in the Summary Module of the CMA Master Workbook) with Most Recent Common Ancestors (MRCAs) from the Target Individual's pedigree. Up to 26 entries may be made in the T° [symbol] starting at cell (EL7). FIG. 8 presents a sample family tree with its corresponding T° [symbol] entries, identified by the surnames of each MRCA couple from the Target Individual's maternal or paternal pedigree. One complex in the example (“Mardell”) is identified by a single surname because we do not have definitive information regarding that ancestor's pedigree.
\textcircled{4} With only the Target Individual's atDNA matches imported, our analytic core set will necessarily be empty ( [symbol] =Ø). A 2^{nd} test subject is required to initiate CMA, with additional subjects required to facilitate comprehensive analysis. Ideally, test subject B should be the closest atDNA match of 1,800 cM or less from subject A's selected (maternal or paternal) ancestral line. Users can [copy/paste special] the name of this match from subject A's atDNA matches into cell (E3), from whence subject B's particulars will be used to populate the white line items in cells (E7:F7) with subject B's name and linkage of [Self]. Next, from the dropdown menu in the field below subject B's name, select the MRCA couple and shared by subjects A and B. (Likewise, in the field below subject A, select the MRCA couple containing subject A, zero generations removed). The user should next copy and paste up to 50,000 of subject B's atDNA matches into the shaded area beginning at cell (E8).
\textcircled{5} FIG. 7 shows the worksheet formula associated with cell (G8) and filled downwards over each of subject B's (up to) 50,000 atDNA matches. The formula itself reads the name or test kit identifier of the first of B's atDNA matches and searches for that same identifier among the range of subject A's matches. The formula also searches for B's identifier amongst the 20,000 possible elements of the analytic core set ( [symbol] ). If the formula finds a match for B's identifier amongst A's atDNA matches but not within [symbol] , then the formula identifies B's identifier as a [Possible add]; otherwise the cell is left blank. This formula returns its result as a [Possible add] because some atDNA services, most notably Ancestry, do not display unique member identifiers among atDNA matches, so the “John Smith” that matches subject A may not necessarily be the individual with the same name who matches B. Users familiar with the Ancestry site have developed methods of identifying these spurious matches.

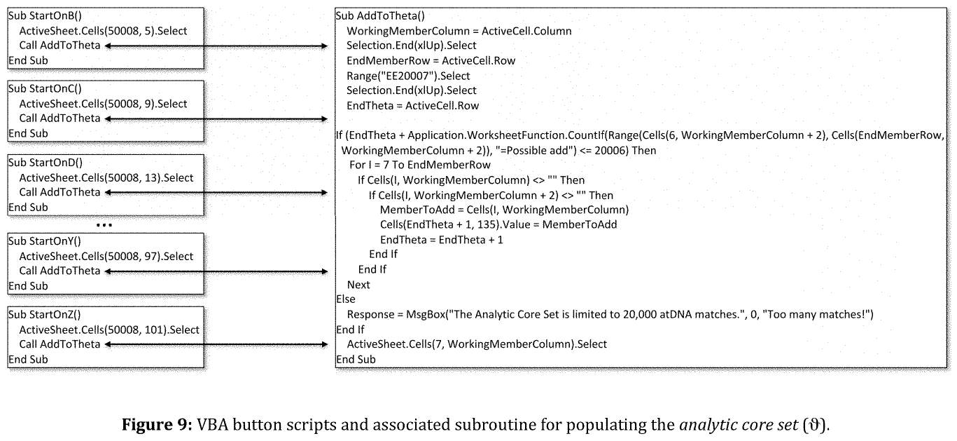

The formula in the scripted button at cell (E5) counts B's atDNA matches and also displays the number of [Possible add]s. If any [Possible add]s exist, clicking on the button at (E5) appends each [Possible add] member to [symbol] . FIG. 9 documents the basic VBA (Visual Basic for Applications) script for each test subject's (row 5) button (StartOnB( ), StartOnC( ), etc.) as well as the common subroutine AddToTheta( ), which populates the analytic core set ( [symbol] ) with a given subject's matches.

The formula in FIG. 7 attached to cell (K8) of subject C is similar to the formula attached to cell (G8) of subject B in that it similarly flags [Possible add]s but subject C's formula checks each of subject C's atDNA matches against the entries of both subjects A and B, returning a [Possible add] only if C's atDNA identifier matches an entry in A or B that does not already appear in [symbol] .

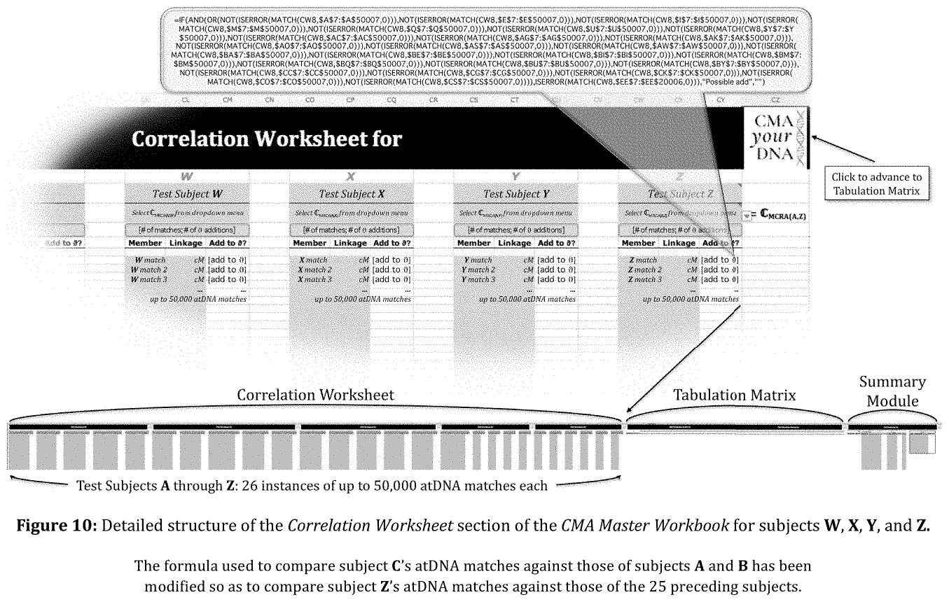

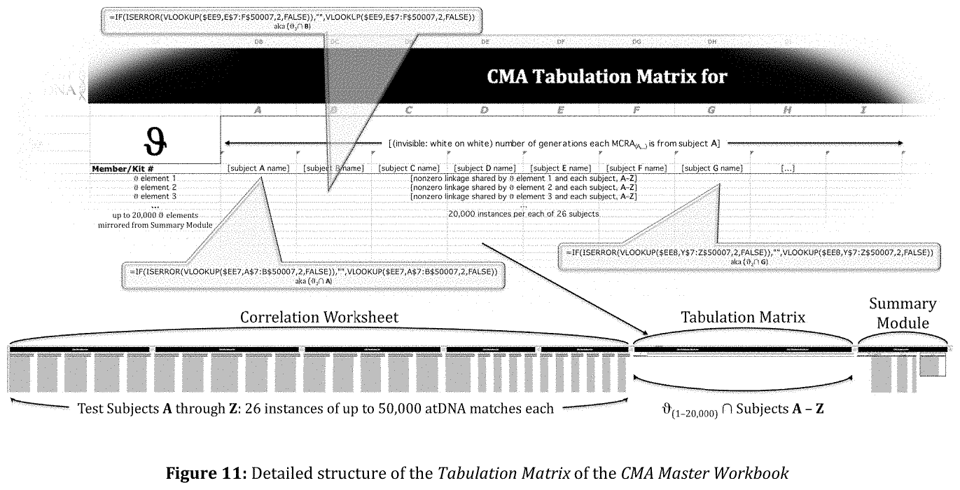

As one might expect, searching for atDNA matches among additional test subjects (C, D, E . . . Y, Z) necessitates a formula that grows increasingly unwieldy. FIG. 10 illustrates that although subject Z's [add to [symbol] ] formula has become gargantuan, its premise remains the same: check each of Z's atDNA identifiers against those of subjects A through Y, and if any such matches are not also found amongst elements of [symbol] , then flag that identifier as a [Possible add].
\textcircled{6}, \textcircled{7}, \textcircled{8} When an MRCA couple from the table of complexes (T° [symbol] ) is assigned to subject B in process \textcircled{4}, an invisible (white on white) field in the Tabulation Matrix stores the number of generations the MRCA couple is removed from the target subject A. FIG. 11 indicates that these fields are embedded in the area between the [symbol] letter-name identifiers of each test subject and the subject's [Member/Kit #] identifier.
\textcircled{9}, \textcircled{10}, \textcircled{1}\textcircled{1} Whenever possible, test subjects should be selected from the Target Individual's atDNA matches with preference given to matches with known genealogical relations to A, to matches with significant linkage, and matches with extensive family trees verified by research and/or shared DNA. A specific line of inquiry may be furthered by the selection or omission of particular test subjects, but the nature of the CMA process is such that so long as the relevant test subjects are eventually included in the set of correlated test subjects, no cumulative difference emerges.

If a newly added test subject's atDNA matches yield a zero value in the number of [Possible add]s it may be that an NPE (non-paternity event) has caused the subject's genetic pedigree to diverge from their presumed genealogical connection to subject A. It's also possible that the newly added subject is the direct descendant of a previous test subject, in which case all of the new subject's connections to A are already manifest in their parent's atDNA matches. A biological child whose atDNA profile matches A in ways their parent does not suggests that both of the child's parents may related to A, which is a significant finding. A test subject only distantly related to A may not show significant correlation until subjects of intermediary relation are analysed, but the Correlation Worksheet allows for any test subject to be removed or replaced without reinitializing the CMA process.
\textcircled{1}\textcircled{2} Each test subject's atDNA matches are subject to the same scrutiny as those of subjects B, C, etc., and as such, [symbol] is progressively augmented with each set of atDNA matches appended to the CMA process. With the exception of the removal of a test subject, the cardinality of [symbol] never reduced.

The leftmost column of the Tabulation Matrix lists individual elements of [symbol] , the analytic core set. These elements are in actual fact mirrored from the ordering of [symbol] displayed in the Summary Module, as these two sections and their data are intimately related. The Tabulation Matrix displays the extent to which each element of [symbol] matches (or does not match) subjects A through Z, with elements of [symbol] listed vertically and test subjects arranged horizontally by letter name. A square in the grid is defined by its (test subject, [symbol] ) co-ordinates and displays the cM linkage of that test subject with that particular element of [symbol] . Where the subject and the [symbol] element are the same, the matrix displays the [Self] notation from the white rows of the Correlation Worksheet.

In addition to displaying the match distribution of corresponding elements among the test subjects and [symbol] , the Tabulation Matrix functions as an intermediary relational data table between each subject's raw atDNA matches and the Summary Module's broad equivalence classes, contributing much of the “correlation” functionality implied by CMA's name. The Summary Module's formulae draw their data almost exclusively from this matrix.

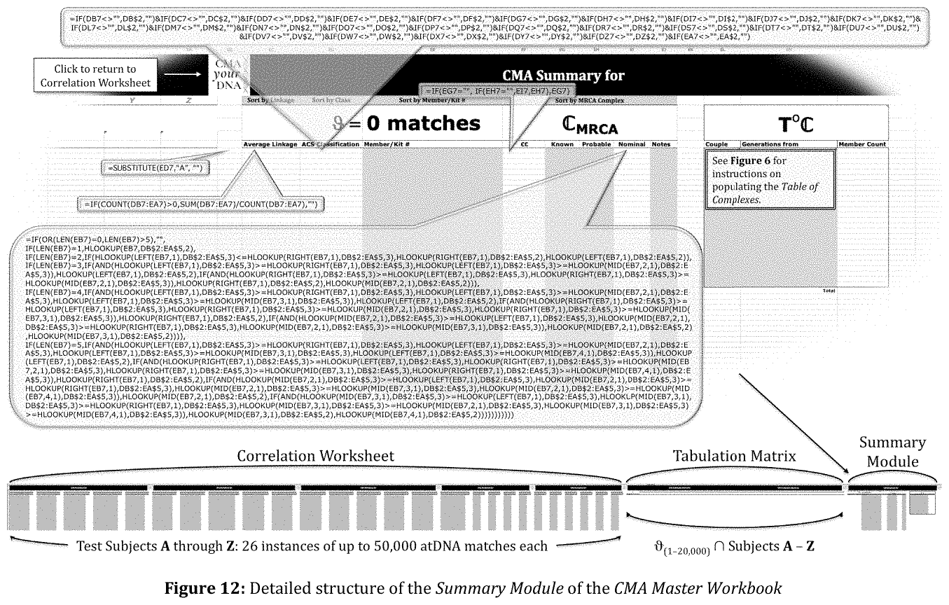

FIG. 12 illustrates the structure of the Summary Module. The leftmost column of the module, “Average Linkage” counts the number of test subjects which match a given element of [symbol] and computes the average linkage shared across those subject matches, providing the user with some statistical shorthand for ranking [symbol] elements within a given class or complex. The CMA Classification (column ED) provides the user with an indispensible measure of the properties of each [symbol] element. The formula classifies each element of [symbol] by harvesting the letter names of the test subjects with which that [symbol] element shares non-zero linkage, regardless of degree. As such, a [symbol] element matching subjects A, D, F, and J would belong to class ADFJ. Sorting by CMA Classification allows us to group together elements of [symbol] which interact similarly with the test subject array, even when we don't precisely know how those elements of [symbol] are connected to the Target Individual and/or the common ancestral lines associated with those [symbol] elements.

Further, CMA Classifications allow the Summary Module to assign a Nominal MRCA-derived genetic complex ( [symbol] _{MRCA(A,x)}) to each member of [symbol] . Because the target test subject A matches the vast majority of elements of [symbol] , and is the reference point from which all MRCA complexes are measured, its presence within a CMA Classification approaches the trivial, and therefore a hidden (white on white) column of formulas (EB) filters the “A” from each CMA Classification prior to assigning it to a complex. The lengthy formula assigned to each cell in column (EI) evaluates a [symbol] element's CMA Classification. If for some reason an element of [symbol] does not match any test subjects, or matches more than 5 matches, no genetic complex ( [symbol] ) is assigned. If the element of [symbol] only matches a single test subject (other than A)—say, x—then [symbol] _{MRCA(A,x)} is assigned. If an element of [symbol] matches 2, 3, 4, or 5 test subjects, the formula examines the constituent letter names within the CMA Classification and compares the number of generations removed from A listed for each letter's MRCA in the T° [symbol] . The letter name with the greatest number of generations removed prevails, and so the element of [symbol] is assigned to [symbol] _{MRCA(A,x)} where x is the letter component of the element's CMA Classification with an MRCA furthest removed from A.

The Nominal Complex assigned to each element of [symbol] represents a computational attempt by the Correlation Worksheet to assign a genetic complex to each element of [symbol] based on an interpretation of available data. However, situations may arise where investigation, deduction, or inference suggests that a [symbol] _{MRCA(A,x)} subset may logically be assigned to another [symbol] —typically a [symbol] further removed from A than computationally assigned. Elements of [symbol] so identified may be provisionally assigned a Probable Complex which may be shown to assume precedence over the Nominal Complex. Lastly, there may be genealogical matches of A whose pedigree and MRCA is well established despite the unavailability of a set of atDNA matches for analysis. These elements of [symbol] can be assigned a Known Complex, taking precedence over the Nominal and Probable assignments. The formula in column (EF), filled down over all elements of [symbol] , assigns this order of precedence to the Known, Probable and Nominal genetic complexes, and it is this Compound Complex ( [symbol] [symbol] ) which is used to sort and stratify elements of [symbol] .

The common matches of two closely related test subjects (say, a half-cousin of A, and that half-cousin's nephew) which share a known MRCA not found in A's pedigree may be labelled according to their probable complex so as to differentiate their abundant matches from the main set of complexes about A's matches. The case study of the Appendix contains such an instance.

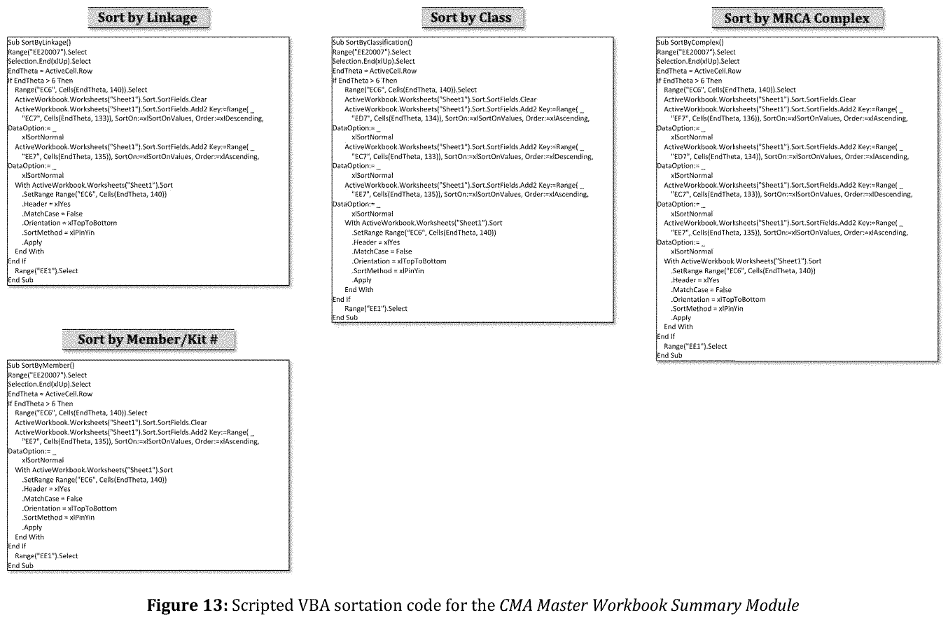

Scripted buttons immediately below the heading bar in FIG. 12 sort the [symbol] elements by Average Linkage only, by CMA Classification (and within each CMA Classification, by Average Linkage), by the name/kit identifier of the [symbol] element, and lastly by MRCA complex (and within each complex by CMA Classification, and by Average Linkage within each CMA Classification). FIG. 13 presents the VBA code behind each of these buttons, which dynamically adjusts the sortation area to accommodate the evolving dimensions of the analytic core set.

Formulae within the table of complexes (T° [symbol] ) tally the number of [symbol] elements in each [symbol] _{MRCA}, and a grand total tracks the number of elements of [symbol] assigned to these complexes.
\textcircled{1}\textcircled{3}, \textcircled{1}\textcircled{5}, \textcircled{1}\textcircled{6} Known genealogical relations of A are assigned an MRCA as from the T° [symbol] as per processes \textcircled{6}, \textcircled{7}, and \textcircled{8}.
\textcircled{1}\textcircled{4}, \textcircled{1}\textcircled{5}^{a} Subjects without a known relation to A may be provisionally designated as a numbered subclass of a known MRCA complex. For instance, if the Correlated Analysis of several test subjects identifies a collection of [symbol] elements about a Common Ancestor John Smith, then the matches shared between [symbol] and first member of this complex added as a CMA test subject may be designated as members of the [symbol] _{Smith-1}. Since all elements of [symbol] _{Smith-1} are also elements of [symbol] _{Smith} we can regard [symbol] _{Smith-1} as a proper subset of its parent, and optionally initiate a second Correlation Worksheet with [symbol] _{Smith} as our new ACS. Otherwise, we can continue to work with numbered subsamples of [symbol] _{Smith} until such time as we are able to identify ancestors common to both A and elements of [symbol] _{Smith}. Sub complexes built from member matches which themselves reside within a subcomplex are identified by a latter suffix ( [symbol] _{Smith-1a}, [symbol] _{Smith-1b}, etc.)
\textcircled{1}\textcircled{6}^{a} The existence of an atDNA test subject whose matches fall completely outside the framework of A's genealogically established common ancestors suggests the presence of an NPE in A's ancestral pedigree, and may be provisionally labeled as an NPEC, until such time as the CMA of other subjects sharing this complex suggests the presence of a common ancestral line. At that point, it may be advisable to suspend CMA until the NPE is resolved and a new set of Common Ancestors with which to populate the T° [symbol] has been assembled.
\textcircled{1}\textcircled{7}, \textcircled{1}\textcircled{8} The [Notes] column in the Summary Module facilitates adding remarks and labeling common family lines in emerging complexes and classes of [symbol] elements. These entries remain sorted with their associated [symbol] elements.

The Appendix presents a case study that demonstrates the elegance and utility of the CMA process as deployed via the CMA Master Workbook.

III. CMA at the Enterprise Level

CMA may be performed at the Enterprise level by deploying relational data structures in a manner consistent with the method employed by the CMA Master Workbook on the desktop platform. The specific methodologies and techniques required to add CMA functionality to an existing genealogical database will necessarily depend on the DBMS (database management system) used, but the general framework outlined in this section should provide adequate guidance to the experienced programmer.

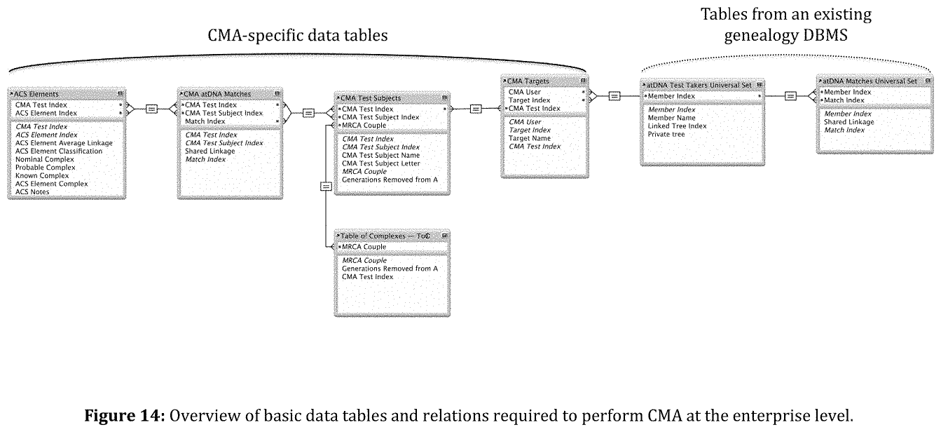

FIG. 14 provides a basic overview of the data tables required to perform CMA at the Enterprise level. Data structures are indicated in Geneva type. Unless prefixed with a new [Table:Field] format, :Fields listed in the same paragraph with an empty table prefix may be assumed to be from the table referenced at the start of the paragraph.

As with Section II, the numbering of processes in the process flowchart of FIG. 1 is maintained in the following description of the structure and operation of CMA at the Enterprise level.

CMA queries will typically originate with a Target Individual corresponding to an account holder/test taker listed in a master table of an atDNA testing service's users, here designated as [atDNA Test Takers].
\textcircled{1} The table [atDNA Test Takers] contains references to all users who have taken atDNA tests. The table has been populated with four provisional fields:
   :Member Index—a unique numerical identifier for each atDNA test subject
   :Member Name—the name of the individual who took the atDNA test
   :Linked Tree Index—a unique numerical identifier which connects the test taker with an individual in a tree owned by the Target Individual.
   :Private Tree—a Boolean field to indicate whether the tree associated with the :Linked Tree Index is public or private.

[atDNA Matches Universal Set] collects every user's test results—the atDNA matches between members—and is augmented with new matches every time a new user is added to the [atDNA Test Takers] table. The [atDNA Matches Universal Set] table requires the following fields:
:Source Index—a numerical identifier for the atDNA test subject whose test generated the match.
:Match Index—a numerical identifier for the member whose test matches the source test subject.
:Shared Linkage—the numerical amount of DNA shared between the two subjects in centiMorgans.

Because atDNA matching is symmetric, the linkage of Match_{(A,B)} is identical to Match_{(B,A)}—and as such, a single table with half the number of records can be queried bilaterally:

{([atDNA Matches Universal Set:Source Index], [atDNA Matches Universal Set:Shared Linkage], [atDNA Matches Universal Set:Match Index])|([atDNA Matches Universal Set:Source Index]=[atDNA Test Takers:Member Index])∪([atDNA Matches Universal Set:Match Index]=[atDNA Test Takers:Member Index])}

in order to obtain subject A's full set of atDNA matches (A). Set A provides an initial set of records for the [CMA atDNA Matches] table.
\textcircled{2} The Target Individual's linked tree may be used to determine an effective query, and traditional methods of clustering employed to identify which atDNA matches fall along maternal or paternal lines.
\textcircled{3} Direct ancestors from the Target Individual's pedigree may be used to populate the :MRCA Couple and :Generations Removed from A fields of the [Table of Complexes—To [symbol] ] table.
\textcircled{4} Additional test subjects (B through Z) may be selected from the list of A's atDNA matches. In each case, the “double-query” of database process 0 should be used to append the subject's matches to the [CMA atDNA Matches] table.
\textcircled{5} From a data management perspective, the simplest method of populating the analytic core set ( [symbol] ; or the [ACS Elements] table) is very likely to build a copy of the [symbol] set ( [symbol] ′) from the [CMA atDNA Matches] table every time a letter-named test subject is added or removed from the [CMA Test Subjects] table, and then reconcile the new copy with the old, as [symbol] is the set of all :Subject Index records of this table with cardinality greater than 1.
\textcircled{6}, \textcircled{7}, \textcircled{8} The [CMA Test Subjects] table has an :MRCA Couple field; when this value is assigned to a test subject, formulas and methods attached to the [ACS Elements] table will update the [ACS Elements:Nominal Complex] field.
\textcircled{9}, \textcircled{10}, \textcircled{1}\textcircled{1} Selection of subsequent test subjects, evaluation of the relationship of those atDNA matches to the [symbol] set (the [ACS Elements] table) and whether to accept or discard those matches will be performed through the user interface to the DBMS.
\textcircled{1}\textcircled{2} As with process \textcircled{5}, the simplest way to update the [ACS Elements] table ( [symbol] ) will be to create a provisional [symbol] set from the records in the [CMA atDNA Matches] table and update/reconcile [symbol] to agree with the provisional [symbol] .
\textcircled{1}\textcircled{3}-\textcircled{1}\textcircled{6} The relationships between the Nominal, Probable, and Known complexes in the [ACS Elements] table are carried over from the CMA Master Workbook, with the calculations populating the :ACS Element Complex giving preference to values in the :Probable Complex field over those of the :Nominal Complex and the :Known Complex taking precedence over all others.
\textcircled{1}\textcircled{7}, \textcircled{1}\textcircled{8} Traditional genealogical research methods have their place in augmenting and interpreting the findings of CMA; this remains the case whether the CMA is performed at the desktop or via a web interface to a DBMS.

REFERENCED CITED

| | | | | |
|---|---|---|---|---|
| Publication # | Priority Date | Publication Date | Asignee | Title |

U.S. Patent Documents

| | | | | |
|---|---|---|---|---|
| 20170213127A1 | 2016 Jan. 24 | 2017 Jul. 27 | Matthew Charles Duncan | Method and System for |
| | | | | Discovering Ancestors using |
| | | | | Genomic and Genealogic Data |
| 20180189379A1 | 2016 Dec. 29 | 2018 Jul. 5 | Ancestry.Com Operations Inc. | Dynamically-qualified aggregate |
| | | | | relationship system in |
| | | | | genealogical databases |
| 10720229B2 | 2014 Oct. 14 | 2020 Jul. 21 | Ancestry.Com DNA, LLC | Reducing error in predicted |
| | | | | genetic relationships |
| 8738297B2 | 2001 Mar. 30 | 2014 May 27 | Ancestry.Com DNA, LLC | Method for molecular |
| | | | | genealogical research |
| 20060025929A1 | 2004 Jul. 30 | 2006 Feb. 2 | Chris Eglington | Method of determining a genetic |
| | | | | relationship to at least one |
| | | | | individual in a group of famous |
| | | | | individuals using a combination |
| | | | | of genetic markers |
| 20090118131A1 | 2008 Oct. 15 | 2009 May 7 | 23andme Inc. | Genetic comparisons between |
| | | | | grandparents and |
| | | | | grandchildren |
| 20140006433A1 | 2013 Apr. 26 | 2014 Jan. 2 | 23andme Inc. | Finding relatives in a database |
| 20140067355A1 | 2013 Sep. 6 | 2014 Mar. 6 | Ancestry.Com DNA, LLC | Using Haplotypes to Infer |
| | | | | Ancestral Origins for Recently |
| | | | | Admixed Individuals |
| 20140108527A1 | 2012 Oct. 17 | 2014 Apr. 17 | Fabric Media Inc | Social genetics network for |
| | | | | providing personal and business |
| | | | | services |
| 20140278138A1 | 2013 Mar. 15 | 2014 Sep. 18 | Ancestry.Com DNA, LLC | Family Networks |
| 8855935B2 | 2006 Oct. 2 | 2014 Oct. 7 | Ancestry.Com DNA, LLC | Method and system for |
| | | | | displaying genetic and |
| | | | | genealogical data |
| 20140067280A1 | 2012 Aug. 28 | 2014 Mar. 6 | Inova Health System | Ancestral-Specific Reference |
| | | | | Genomes And Uses Thereof |

Foreign Patent Documents

| | | | | |
|---|---|---|---|---|
| WO2019217574A1 | 2018 May 8 | 2019 Nov. 14 | Ancestry.Com Operations Inc. | Genealogy item ranking and |
| | | | | recommendation |
| W02020018991A1 | 2018 Jul. 20 | 2020 Jan. 23 | Ancestry.Com Operations Inc. | System and method for |
| | | | | genealogical entity resolution |
| W02020257166A1 | 2019 Jun. 17 | 2020 Dec. 24 | Ancestry.Com Operations Inc. | Genealogical tree tracing and |
| | | | | story generation |
| W02021051018A1 | 2019 Sep. 13 | 2021 Mar. 18 | 23andme, Inc. | Methods and systems for |
| | | | | determining and displaying |
| | | | | pedigrees |
| W02000018960A3 | 1998 Sep. 25 | 2000 Sep. 8 | Ancestry.Com DNA, LLC | Methods and products related |
| | | | | to genotyping and DNA analysis |
| W02009051766A1 | 2007 Oct. 15 | 2009 Apr. 23 | 23andme, Inc. | Family inheritance |

1. A process for performing Correlated Multiphasic Analysis (CMA) of autosomal DNA (atDNA) matches, independent of any specific testing provider or tabulating mechanism.

2. The process of claim 1, where the atDNA matches of a Target Individual are logically compounded with the matches of other test subjects via unary operations including, but not limited to: intersection, union, and complementation.

3. The process of claim 1, whereby additional test subjects are selected from the atDNA matches of the Target Individual based on criteria including, but not limited to:
a) the ancestral family line shared by the Target Individual and test subject.
b) the amount of atDNA linkage shared by the Target Individual and test subject.
c) test subjects with extensive family trees verified by research and/or DNA.
d) test subjects whose shared linkage with the Target Individual ranks them at the top of their genetic complex.
e) test subjects whose atDNA may contain specific markers for biological traits or genetic predispositions relevant to epidemiology or genetic counseling.

4. The process of claim 1, whereby an analytic core set (variously ACS, or [symbol] ) is compounded from the logical intersection of the atDNA matches of dyads of test subjects.

5. The process of claim 1, whereby the analytic core set is cross-referenced against a roster of test subjects to generate a CMA Classification consisting of letter-name identifiers associated with each test subject.

6. The process of claim 1, whereby the CMA Classification of each element of the ACS is parsed to assign each element to a genetic complex ( [symbol] ).

7. The process of claim 1, whereby a genetic complex is the set of all individuals whose atDNA matches any two members of a collection of test subjects sharing an MRCA couple.

8. The process of claim 1, whereby genetic complexes are labeled according to the surnames of the MRCA couple common to the test subjects which populate that complex (i.e. [symbol] _{[Smith-Jones]}).

9. The process of claim 1, whereby genetic complexes are tallied in a Table of Complexes (T° [symbol] ) consisting of MRCA couples taken from the Target Individual's pedigree alongside their “generation number”—the number of generations each MRCA couple is removed from the Target Individual.

10. The process of claim 1, wherein parsing the CMA Classification of an element of the ACS entails comparing the generation numbers of the MRCA couples of each letter-name in the CMA Classification and assigning that element of the ACS to the nominal genetic complex defined by the MRCA couple with the greatest generation number.

11. Scripted spreadsheet implementations of the process of claim 1.

12. Spreadsheet implementations of claim 11, wherein a tripartite arrangement of related data structures performs CMA via correlation, tabulation and summary.

13. Spreadsheet implementations of claim 11, wherein the construction of the analytic core set entails compounding the intersection sets of dyads of sets of atDNA matches from test subjects.

14. Spreadsheet implementations of claim 11, wherein the progressive cyclical compounding of test subject dyads entails comparing each element within a set of atDNA matches against the entirety of previously added sets.

15. Spreadsheet implementations of claim 11, wherein individual additions to the analytic core set flagged for processing are tallied by test subject and displayed within the label of a scripted button alongside a census of a test subject's atDNA matches.

16. Spreadsheet implementations of claim 11, wherein the user populates a Table of Complexes (T° [symbol] ) with ancestral couples from the Target Individual's pedigree and their associated “generation number”—a natural number equal to the number of generations each couple is removed form the Target Individual.

17. Spreadsheet implementations of claim 11, wherein the CMA Classification assigned to each element of the analytic core set by the Summary Module is a concatenation of the letter-name identifiers of the test subjects which share atDNA with that element of the analytic core set.

18. Spreadsheet implementations of claim 11, wherein the formulation of a Nominal Complex for an element of the analytic core set by the Summary Module necessitates segmenting an element's CMA Classification into individual test subject letter-names and evaluating the “generation number” associated with the MRCA/complex of each letter-name, such that the letter-name with the greatest “generation number” establishes the value of the Nominal Complex.

19. A DBMS (Database Management System) implementation of the process of claim 1.

20. The DBMS implementation of claim 19, wherein CMA-specific data tables and methods are appended to an existing genealogical DBMS.
